(19) United States
(12) Patent Application Publication
(10) Pub. No.: US 2023/0338147 A1
(43) Pub. Date: Oct. 26, 2023

(54) FRAME RELEASE MECHANISM FOR VALVE REPAIR DEVICES

(71) Applicant: Boston Scientific Scimed, Inc., Maple Grove, MN (US)

(72) Inventors: Graham Krumpelmann, Stillwater, MN (US); Joshua M. Inouye, Maple Grove, MN (US); James M. Anderson, Corcoran, MN (US)

(73) Assignee: Boston Scientific Scimed, Inc., Maple Grove, MN (US)

(21) Appl. No.: 17/781,774

(22) PCT Filed: Aug. 5, 2020
(86) PCT No.: PCT/US2020/044924

§ 371 Date: Jun. 2, 2022

Related U.S. Application Data
(60) Provisional application No. 62/882,640, filed on Aug. 5, 2019.

(51) Int. Cl. A61F 2/24

(52) U.S. Cl. CPC A61F 2/2466; A61F 2220/0016

(57) ABSTRACT

A low profile implant, system and method of deployment includes a plurality of anchors supported by a plurality of anchor housings. The anchor housings include one or more release mechanisms configured to affix the frame to the anchor housings during implant deployment, actuation and cinching, and to release the frame following cinching to enable the frame to be withdrawn from the treatment site.

The present application claims the benefit of priority under 35 U.S.C. § 119 to U.S. Provisional Patent Application 62/882,640, filed Aug. 5, 2019, which application is incorporated herein by reference in its entirety for all purposes.

### FIELD

The present disclosure relates generally to the field of implantable medical devices. In particular, the present disclosure relates to medical devices, systems, and methods for annuloplasty and other cardiac treatment techniques.

### BACKGROUND

Mitral insufficiency (MI) (also referred to as mitral regurgitation or mitral incompetence) is a form of heart disease where the mitral annulus dilates excessively and the valve leaflets no longer effectively close, or coapt, during systolic contraction. Regurgitation of blood occurs during ventricular contraction and cardiac output may decrease as a result. Surgical and endoluminal annuloplasty techniques have been introduced that aim to restore a mitral valve to its native configuration, for example by implanting an annuloplasty ring around a valve annulus. One problem encountered by such implants is that their size may cause unintended contact between the implant and the cardiac wall, reducing the efficacy of the implant. It is desirable to minimize the size of an implant to reduce the opportunity for such contact and it is with these considerations in mind that the improvements of the present disclosure may be useful.

### SUMMARY

Embodiments of the present disclosure relate to a system, device, and method for reshaping a valve annulus such as a heart valve annulus.

According to one aspect, a system for annuloplasty includes a frame having a proximal end, a distal end and struts joined at one of the proximal end or the distal end of the frame, and an implant. The implant may include a plurality of anchors and a plurality of anchor housings, at least one anchor housing configured to support and retain one of the plurality of anchors, the at least one anchor housing including a release mechanism configured to releasably couple the at least one anchor housing to the frame.

In one embodiment the at least one anchor housing may include a bore extending from a proximal end of the at least one anchor housing through the distal end of the at least one anchor housing and a slot extending longitudinally through a wall of the bore. The release mechanism may include a retention tab disposed within the slot. In one embodiment, the retention tab may include a distal foot, a proximal head and an arm extending from the distal foot to the proximal head. The retention tab may include a first configuration where an internal face of the proximal head extends into the bore and a second configuration where the internal face of the proximal head is aligned with the wall of the bore. The bore may be sized to accept an anchor and the anchor may include a drive shaft including a proximal connector, a distal anchor shaft coupled to the proximal connector and a collar extending at least partially around the distal anchor shaft. In some embodiments, the bore and the collar may be sized to enable distal translation of the collar within the bore of the anchor housing. A first diameter of the proximal connector may be less than a second diameter of the collar and distal translation of the collar beyond the proximal head of the retention tab may return the proximal head towards the second configuration to retain the anchor in the anchor housing. In one embodiment, the proximal head of the retention tab may include a frame engagement feature disposed on an exterior face of the proximal head. The frame engagement feature may be sized to mate with an anchor housing engagement feature of the frame. In the first configuration of the retention tab the frame engagement feature may engage the anchor housing engagement feature, and in the second configuration the frame engagement feature may release the anchor housing engagement feature to release the frame from the implant.

In one embodiment, the release mechanism of the at least one anchor housing may include a primary housing coupled to the frame, and a secondary housing releasably coupled to the primary housing by an anchor. The primary housing may include a primary bore extending therethrough and a first anchor engagement feature disposed on a primary bore wall. The secondary housing may include a secondary bore extending therethrough and a second anchor engagement feature disposed on a secondary bore wall, the second anchor engagement feature extending further centripetally within the secondary bore than the first anchor engagement feature extends within the primary bore. The primary housing may include a proximal end and a distal end, and the secondary housing may be configured to matingly engage with the distal end of the primary housing to align the primary bore with the secondary bore. In one embodiment, the primary bore, and secondary bore may be sized to support the anchor, and the anchor may include a drive shaft including a proximal connector, a distal anchor shaft and a collar extending at least partially around the distal anchor shaft. The distal anchor shaft may include at least two external engagement features, each configured to engage at least one of the primary anchor engagement feature or the secondary anchor engagement feature. In one embodiment, a first centripetal extent of the first anchor engagement feature may be configured to enable distal advancement of the collar of the anchor into the primary bore, and a second centripetal extent of the second anchor engagement feature may be configured to preclude distal advancement of the collar into the secondary bore

According to a further aspect, an implant includes a plurality of anchors and at least two anchor housings. Each anchor housing includes a release mechanism configured to releasably couple the anchor housing to an implant delivery system. The implant also includes a cinch lumen extending through a portion of the anchor housing and a cinch cord extending through the cinch lumen of the at least two anchor housings to join the at least two anchor housings.

In one embodiment, at least one anchor housing may include a bore extending from a proximal end of the at least one anchor housing through a distal end of the at least one anchor housing and a slot extending longitudinally through a wall of the bore. The release mechanism may include a retention tab disposed within the slot, the retention tab including a distal foot, a proximal head, and an arm extending from the distal foot to the proximal head, where the retention tab may include a first configuration where an internal face of the proximal head extends into the bore and a second configuration where the internal face of the proximal head is aligned with the wall of the bore. In one embodiment, the implant delivery system may include a frame and the proximal head of the retention tab may include a frame engagement feature disposed on an exterior face of the proximal head, the frame engagement feature sized to mate with an anchor housing engagement feature of the frame. In one embodiment, in the first configuration of the retention tab the frame engagement feature may engage the anchor housing engagement feature, and in the second configuration the frame engagement feature may release the anchor housing engagement feature to release the frame from the implant. In one embodiment, the implant delivery system may include a frame, and the release mechanism of the at least one anchor housing may include a primary housing coupled to the frame and a secondary housing releasably coupled to the primary housing by an anchor. The cinch lumen may be provided by the secondary housing.

According to a further aspect, an annuloplasty method includes the steps of deploying an implant system to a valve annulus, the implant system including a frame and an implant including a plurality of anchors translatably supported by a plurality of anchor housings. The plurality of anchor housings is releasably coupled to the frame by a release mechanism of each anchor housing. The method includes the steps of securing the implant to the valve annulus by driving the plurality of anchors at least partially through the plurality of anchor housings and into the valve annulus, cinching the valve annulus using a cinching mechanism of the frame, releasing the frame from the plurality of anchor housings using the release mechanism of each anchor housing, and withdrawing the frame from the valve annulus.

In one embodiment, the method at least one anchor housing may include a bore extending from a proximal end of the at least one anchor housing through a distal end of the at least one anchor housing, and the release mechanism of each anchor housing may include a retention tab disposed within the bore and having a frame engagement feature configured for mated engagement with an anchor engagement feature of the frame when the retention tab is in an unbiased state. The step of releasing the frame from the plurality of anchor housings may include the steps of distally translating an anchor through the at least one anchor housing until the retention tab returns to a biased state to release the frame engagement feature from the anchor engagement feature.

In one embodiment, the at least one anchor housing may include a primary housing coupled to the frame and a secondary housing releasably coupled to the primary housing by an anchor. The release mechanism of each anchor housing may include a first anchor engagement feature disposed within a primary bore of the primary housing and a second anchor engagement feature disposed within a secondary bore of the secondary housing, the second anchor engagement feature extending further centripetally within the second bore than the first anchor engagement feature extends within the primary bore. The step of releasing the frame from the plurality of anchor housings may include the step of distally translating the plurality of anchors through the primary housings and the secondary housings until proximal anchor collars of the plurality of anchors extend into the secondary housing and are precluded from further distal translation by the second anchor engagement feature.

With such an arrangement, a low-profile valve annulus implant with increased flexibility and a reduced potential for inadvertent contact with cardiac tissue is provided.

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical illustrated component is typically represented by a single numeral. For purposes of clarity, not every component is labeled in every figure, nor is every component of each embodiment shown where illustration is not necessary to allow those of ordinary skill in the art to understand the disclosure. In the figures:

FIG. 1 is a diagram of one embodiment of an implant comprising anchor housings as disclosed in various embodiments herein;

FIG. 2A illustrates apices of a frame including anchor housings configured in accordance with an embodiment of the present disclosure;

FIG. 2B is a cross section illustration of the anchor housing of FIG. 2A;

FIG. 2C is a cross-section illustration of one embodiment of a portion of an implant including an anchor housing as disclosed in FIG. 2A;

FIG. 3 is a cross section illustration of one embodiment of a portion of an implant including an anchor housing as disclosed in FIG. 2A, illustrating an anchor advanced through the anchor housing;

FIGS. 4A and 4B illustrate one embodiment of a frame release mechanism and released implant components as disclosed herein;

FIGS. 5A-5D illustrate one embodiment of a modular anchor housing in accordance with the present disclosure;

FIG. 6 illustrates the anchor housing of FIGS. 5A-5D during an exemplary method of deploying an implant;

FIGS. 7A and 7B illustrate one embodiment of a frame release mechanism of an anchor housing in accordance with the present disclosure;

FIGS. 8A-8C illustrate various embodiments of anchor housings including lumens extending therethrough as disclosed herein;

FIGS. 9A-9C illustrate one embodiment of a method for deploying and retrieving annuloplasty components including portions of anchor housings as disclosed herein; and

FIG. 10 illustrates an exemplary handle embodiment that may be used to control deployment of an implant including release of a frame as disclosed herein.

### DETAILED DESCRIPTION

A low profile implant, system, and method of deployment, as disclosed herein in various embodiments, includes a generally tubular frame having a proximal end and a distal end. In one embodiment, the frame is configured for custom reshaping of the heart valve. The distal end of the frame includes one or more anchor housings, each anchor housing carrying an anchor that may be translatably advanced through the anchor housing and into tissue at a treatment site. In various embodiments, the anchor housing may include a lumen extending through at least a portion of the anchor housing, the lumen configured to support a cinch cord that binds anchor housings together to control and/or to retain the custom reshaping of the heart valve.

According to one aspect, each anchor housing may include one or more release mechanisms configured to affix the frame to the anchor housing during implant deployment and heart valve reshaping, and to release the frame following heart valve reshaping to allow removal of the frame from the treatment site. The anchor housing may also include at least one retention mechanism, configured to secure the anchor within the anchor housing following deployment of an anchor into tissue. The resulting low profile implant reduces the potential of inadvertent contact between the implant and the cardiac wall and the associated side effects and enables a lighter weight frame material to be used, since the frame is removed and therefore need not be built for chronic use.

These and other beneficial aspects of an implant and method of deployment are described in more detail below. Although embodiments of the present disclosure may be described with specific reference to mitral valves, the principles disclosed herein may be readily adapted to facilitate reconstruction of any valve annulus, for example including a tricuspid valve annulus and/or may similarly benefit any other dilatation, valve incompetency, valve leakage and other similar heart failure conditions.

As used herein, the term “distal” refers to the end farthest away from the medical professional when introducing a medical device into a patient, while the term “proximal” refers to the end closest to the medical professional when introducing a medical device into a patient.

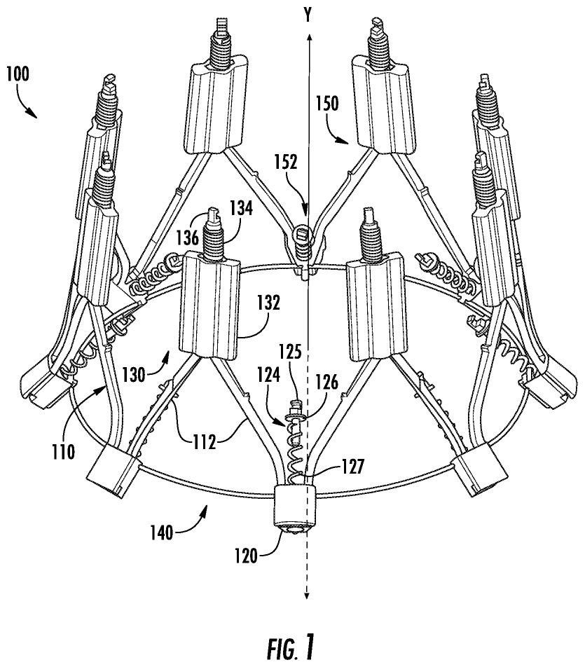

FIG. 1 illustrates an implant 100 comprising a frame 110 that may be disposed about a heart valve or other cardiac feature. For purposes of clarity not all of the components of the implant are numbered. In one embodiment, the frame 110 may extend circumferentially around and partially axially along a central frame axis Y extending proximally-distally through a center point of the frame. The frame 110 may be generally symmetric with respect to the central frame axis although it need not be symmetric. The frame 110 may form a generally tubular shape, where herein “tubular” includes circular as well as other rounded or otherwise closed shapes. The frame 110 may be configured to change shape, size and/or configuration. For example, the frame 110 may assume various shapes, sizes, configurations etc. during different phases of deployment such as during pre-delivery, delivery, tissue engagement and cinching.

According to one embodiment, the frame 110 may be formed from one or more struts 112 that may form all or part of the frame 110, where the struts 112 may include elongated structural members formed of a metal alloy, a shape memory material, such as an alloy of nickel titanium or other metals, metal alloys, plastics, polymers, composites, other suitable materials, or combinations thereof. In FIG. 1 sixteen struts 112 are shown although it is appreciated that in some embodiments, there may be fewer or more than sixteen struts.

In one embodiment, the struts 112 of the frame 110 may be formed from the same, monolithic piece of material (e.g. tube stock). Thus, reference to struts 112 may refer to different portions of the same, extensive component. Alternatively, reference to struts 112 may refer to components that are formed separately and attached permanently together, for example by welding or other methods. In some embodiments, the struts 112 may be separate components that are detachably coupled to form proximal apices 150 and distal apices 152. For example, the struts 112 are shown joined at their proximal apex by actuator 130 and at their distal apex by anchor housings 120.

In some embodiments, the terms “apex,” apices,” and the like may be used interchangeably with terms “crown,” “crowns,” and the like, as used herein and as used in any reference incorporated by reference herein, unless otherwise stated. In one embodiment, an ‘apex’ may include a proximal or distal portion of the frame.

In one embodiment, the actuator 130 includes an actuator shaft 134 that is rotatably carried by the proximal end of the frame 110. For example, a head of the actuator shaft 134 may be contained within the actuator collar 132 and be carried by a window or other opening (not shown) at the proximal apex 150 of the frame 110 to enable rotation of the shaft 134 within the actuator collar 132. The actuator shaft 134 may include a drive coupler 136 disposed at a proximal end.

The actuator collar 132 may include internal features configured to interact with the features of the actuator shaft 134 such that rotation of the actuator shaft 134 by an actuator drive tube coupled to the drive coupler 136 axially translates the actuator collar 132 over the actuator shaft 134 and over struts 112. In some embodiments, “axial” as applied to axial movement or restraint of the actuator collar includes those directions that are at least partially in the proximal or distal direction and that are parallel or generally parallel to a central axis extending through (e.g. proximally—distally) the frame. As shown in FIG. 1, struts 112 extend away from the proximal apex in opposing directions. Distal translation of the actuator collar 132 pulls struts 112 together within the actuator collar 132, thereby reducing the distance between anchor housings 120 for annular customization. Actuator collar 132 may be independently actuated in accordance with the reshaping objective for the associated anchor pair.

The implant further includes anchor housings 120 coupled by a cinch cord 140. As described below in various embodiments, the anchor housings may include one or more release mechanisms configured to release the frame 110 from at least a portion of the anchor housing 120. The anchor housings 120 may also include one or more retention mechanisms configured to retain the anchor 124 within the anchor housings 120. In some embodiments, the cinch cord 140 may be made of nylon or other suture material and used to retain and/or change relative positions of the anchor housings 120 prior or subsequent to release of the frame 110 from the anchor housings 120.

Each anchor housing 120 carries an anchor 124 having a proximal head 126 coupled to an anchor shaft 127, shown to comprise a helical shaft in FIG. 1. A drive coupler 125, disposed at the proximal head of the anchor 124 is configured to cooperate with a complementary feature of a drive tube (not shown) to axially translate the anchor 124 through the anchor housing 120 and into tissue. As described in more detail below, once the anchors 124 are driven through the anchor housings 120 and into tissue, the frame 110 may be released from at least a portion of the anchor housing 120.

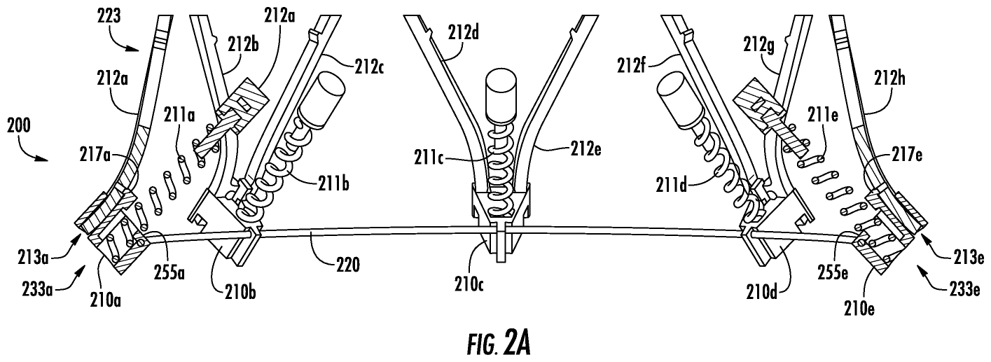

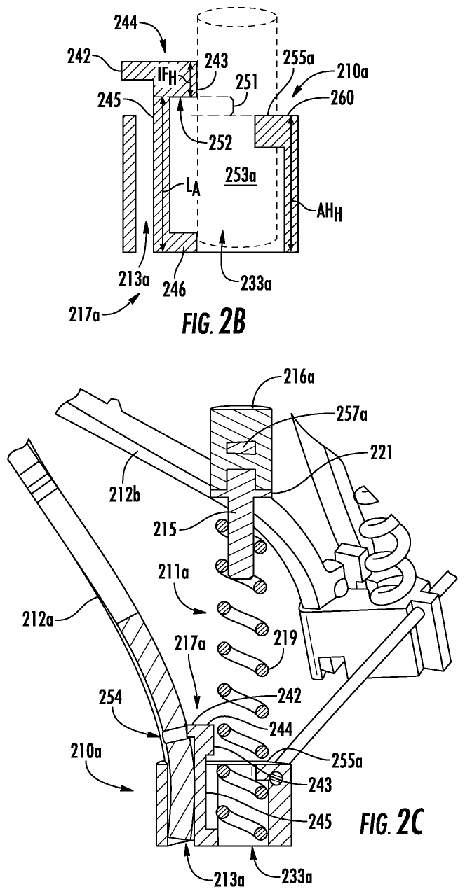

FIG. 2A illustrates a section of one embodiment of a customizable, low profile implant 200, wherein struts 212a, 212h, anchors 211a, 211e and anchor housings 210a, 210e are shown in cross section. Implant 200 includes a frame 223 comprising a plurality of struts 212a-212h. Adjacent struts are shown coupled at distal ends via anchor housings 210a-210e, for example strut 212a is disposed within anchor head sleeve 213a, and strut 212h is disposed within anchor head sleeve 213h. In one embodiment, the anchor housing 210a is configured to retain the strut 212a during deployment, release the strut 212a following deployment and/or secure a deployed anchor 211a to the anchor housing 210a following tissue engagement. Anchor housings may also include lumens extending therethrough for slideably supporting a cinch cord 220 for adjusting/securing the implant. In FIG. 2A, anchor housings 210a and 210e are shown in cross section to highlight retention tabs 217a, 217e. In one embodiment, retention tabs 217a, 217e may serve the dual purpose of allowing retention/release of the frame from the anchor housing and securing the anchor to the anchor housing. Providing frame retention/release mechanisms as part of the anchor housing enables the frame to be beneficially used to customize an annular implant according to the particular needs of a patient and/or diseased valve and later released to minimize an implant profile. Providing anchor securing mechanisms in the anchor housing reduces issues associated with anchor migration and backout during chronic use.

According to one aspect, each anchor housing 210a-210e includes a bore, such as bore 233a, 233e, matched generally in circumference to the outer diameter of an anchor, through which anchors 211a-211e may be translated to engage tissue. Each bore 233a, 233e may include anchor engagement features, such as threads, ridges, cuts, etc. disposed on a bore wall. The anchor engagement features may interact with the anchor to translate the anchor through the anchor housing, for example, engagement features disposed on a wall of bore 233a may include ridges along which turns of anchor 211a may ride during rotation of a driver 216a of anchor 211a to provide distal or proximal translation of the anchor. According to one aspect, each anchor housing may include an anchor engagement feature such as ledges 255a, 255e, extending at least partially into a proximal end of the bore. In various embodiments as described in more detail below, the ledges 255a, 255e serve both as an engagement feature, guiding advancement of the anchor through the bore, and a retention feature which precludes advancement of an anchor retention feature into the bore to enable the anchor retention feature to be locked to the anchor housing as described below. Retention tabs 217a, 217e may be disposed within a slot of the bore.

Retention tab 217a is shown in cross section in more detail in FIG. 2B to include a distal foot 246, which may be affixed to or integral with the anchor housing 210a. Retention tab 217a further includes a proximal head 244, extending above a proximal surface 260 of the anchor housing 210a and a resilient arm 245 joining the distal foot 246 to the proximal head 244. In one embodiment, the resilient arm 245 may be comprised of a memory metal, such as Nitinol, spring tempered stainless steel, PEEK plastics or other strong, flexible material or combination of materials. In one embodiment, the resilient arm may be biased towards a central axis of the bore 233a and positioned within a slot of the bore for free movement of the arm 245 between an unbiased position as shown in FIG. 2B and a biased position where the proximal head 244 interferes with the spatial volume 253a defined by bore 233a. As described in more detail below, interference of the proximal head 244 with the spatial volume 253a as defined by the bore 233a may lock an anchor within the bore 233a to prevent anchor backout during use.

According to one aspect, the proximal head 244 may include features adapted for anchor locking. For example, the proximal head 244 may include an internal face 243 oriented towards a central axis of the bore 233a of the anchor housing 210a. The internal face 243 may have an internal face height (IFH) selected in response to a spacing between threads of the anchor. The internal face 243 may cooperate with a retention feature of an anchor to retain the anchor within the anchor housing 210a.

For example, FIG. 2C illustrates an anchor 211a having a proximal shaft 215 including a drive coupler 257 shown coupled to driver 216a. A collar 221 extends radially from the proximal shaft 215, and a helical shaft 219 is shown coupled to the proximal shaft 215. The anchor 211a is shown extending through the bore 233a of anchor housing 210a. The IFH of the proximal head 244 of the retention arm 217a may be equal to or exceed the maximum spacing between turns of a helical shaft 219, allowing the internal face 243 of the proximal head 244 of the retention arm 217a to ride along the exterior surface of the helical shaft 219 without falling between turns of the helical shaft and interfering with anchor translation.

In various embodiments the anchors, such as anchor 211a, may be made of a suitable biocompatible metal alloy such as stainless steel, cobalt chromium, platinum iridium, nickel titanium, other suitable materials, or combinations thereof. Each anchor may be sharpened at its distal point, or leading turn, so as to facilitate penetration into the cardiac tissue. Each anchor may be from about ten to about fifteen millimeters (mm) in total axial length. In some embodiments, the anchors may be shorter or longer than ten to fifteen millimeters (mm) in total axial length. By “total” axial length it is meant the axial length of the anchor from the end of the distal penetrating tip to the opposite, proximal end of drive coupler 257. The helical shaft 219 may be from about six to about twelve millimeters (mm) in axial length, i.e. in an axial direction. In some embodiments, the helical portion of the anchor may be shorter or longer than six to twelve millimeters (mm) in axial length. The anchor head and/or other non-helical portions of the anchor may be from about three to about four millimeters (mm) in axial length. In some embodiments, the helical diameter range may extend from (0.050″-0.080″), and pitch from (0.030″-0.080″), such that the coil pitch angle is about twenty (20) degrees.

In one embodiment, the collar 221 provides the retention feature of the anchor 211a that cooperates with the internal face 243 of the proximal head 244 of the resilient arm 217a to lock the anchor 211a to the anchor housing 210a following deployment of anchor 211a into tissue. In one embodiment, a diameter of the collar 221 relates to an outer diameter of the helical shaft 219. In some embodiments, the diameter of the collar 221 is matched to the outer diameter of the helical shaft 219. In some embodiments the drive coupler 257a has a diameter that is smaller than the collar 221. For example, as shown in FIG. 2C, the diameter of the drive coupler 257a may correspond to the diameter of the drive shaft 215 that extends through the inner diameter of helical shaft 219. An outer diameter of driver 216a may meet or exceed the outer diameter of collar 221.

Referring back to FIG. 2B, in one embodiment a length of the arm LA is selected such that the offset 251 between the distal end of the proximal head 244 of the retention arm 217a and the proximal surface 260 of the anchor housing 210a is at least equal to the thickness of a retention feature of the anchor. For example, in embodiments wherein the retention feature of the anchor comprises the collar 221 (FIG. 2C), the offset is selected to enable the collar 221a to be retained between the ledge 255a formed by the proximal surface 260 of the anchor housing 210a, and the distal surface 252 of the proximal head 244 of the retention tab 217a when the arm 245 is moved to a biased position and the proximal head 244 is advanced towards the central axis of the bore. Accordingly, the arm length LA may be at least equal to the length of the height of the anchor housing (AHH) plus the thickness of the anchor retention feature. In other words, the offset 251 may be greater than or equal to the thickness of the anchor retention feature.

In addition to anchor retention features, in some embodiments, the proximal head 244 may further include frame engagement features adapted for mating with an anchor housing engagement feature of a frame for frame retention and/or release. For example, the proximal head 244 may include a protuberance such as lip 242 disposed on an external surface of the proximal head, wherein ‘internal’ or ‘interior’ refers to a surface of the proximal head oriented towards the bore, and an ‘external’ or ‘exterior’ surface is a surface other than the internal surface. In one embodiment, the lip 242 is sized to slideably engage a slot on a strut of a frame that extends through anchor housing sleeve 213a in anchor housing 210a.

For example, FIG. 2C illustrates a strut 212a disposed in anchor housing sleeve 213a and including a slot 254 adapted to slideably engage the lip 242 of the proximal head 244 of the retention arm 217a. When so engaged, lip 242 retains the strut 212a in sleeve 213a, to couple the strut 212a to the housing 210a. In alternate embodiments, the external surface of the proximal head 244 may include a slot or other opening configured to slideably accept a tab or other protuberance provided on a strut of the frame. In either embodiment, transition of the arm 245 from the unbiased position towards the biased position releases the lip 242 from the slot 254 of the frame, to thereby release the strut 212a from anchor housing sleeve 213a of the housing 210a. In one embodiment, a length of the lip 242 or other protuberance may be selected to ensure that the lip 242 may be fully released from the slot 254 in the strut 212a when the retention arm 217a is in the biased position.

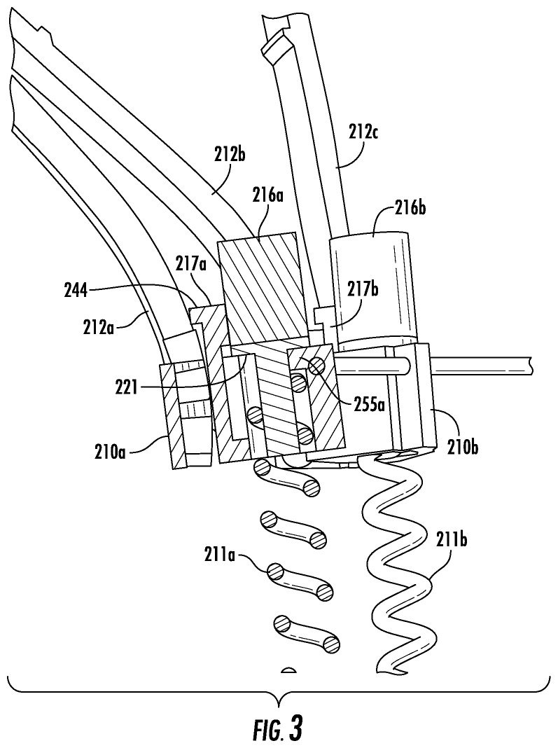

FIG. 3 illustrates anchor housings 210a, 210b coupled to struts 212a, 212b, 212c and supporting distally deployed anchors 211a, 211b. In FIG. 3, anchors 211a, 211b are shown in a tissue engaged configuration, where drivers 216a and 216b have been advanced to translate respective anchors 211a, 211b through the anchor housings 210a, 210b. Translation continues until, for example, the collar 221 of the anchor 211a has been advanced distally past the proximal head 244 of retention tab 217a and ledge 255a which impedes further distal translation of the anchor 211a. The exterior surface of drivers 216a, 216b retain the retention tabs 217a, 217b in their unbiased configuration.

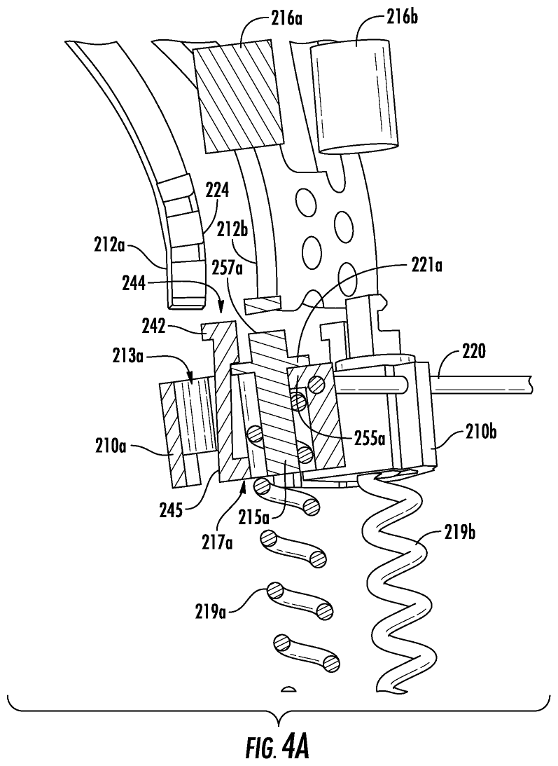

FIG. 4A illustrates anchor housings comprising the frame release and anchor securing mechanisms disclosed above following anchor deployment. In FIG. 4A, anchor housing 210a and associated components are shown in cross section. Following distal advancement of the helical anchors 219a, 219b such that collars 221a, 221b are precluded from further distal advancement by ledges 255a, 255b (not visible), the anchor drivers 216a, 216b may be released from the drive couplers 225a, 225b. Release of the driver 216a from the drive couplers 225a relieves the force on the proximal head 244 that acts against the bias of the retention tab 217a, 217b, allowing the arm 245 of the retention tab 217a to revert to its biased position. In the biased position, shown in FIG. 4A, the proximal head 244 of the retention tab 217a advances towards the central axis of the bore, locking the proximal head 244 over the collar 221 of the shaft 215a to retain the collar 221 between the proximal head 244 of the retention tab 217a and the ledge 255a of the anchor housing.

In one embodiment, advancement of the proximal head 244 centripetally towards the central axis of the bore withdraws the lip 242 from the slot 224 of strut 212a, enabling the strut 212a to be released from sleeve 213a of housing 210a. As a result, a low profile implant consisting only of anchor housings, anchors and cinch cord 220 remains within the cardiac cavity.

FIG. 4B illustrates a portion of implant 200 that may remain after anchors 211a-211e have been driven into tissue 450, for example cardiac tissue, and the frame has been withdrawn from the cardiac cavity. As shown in FIG. 4B only the anchor housings 210a-210e, anchors 211a-211e and cinch cord 220 remain in the cardiac cavity. The height of the resulting implant is reduced to the height of the anchor housings 210a-210e and the proximal extent which drive couplers 257a-257e extend from the anchor housing. As a result, an implant profile may be greatly reduced and the potential for inadvertent contact with heart features may be minimized.

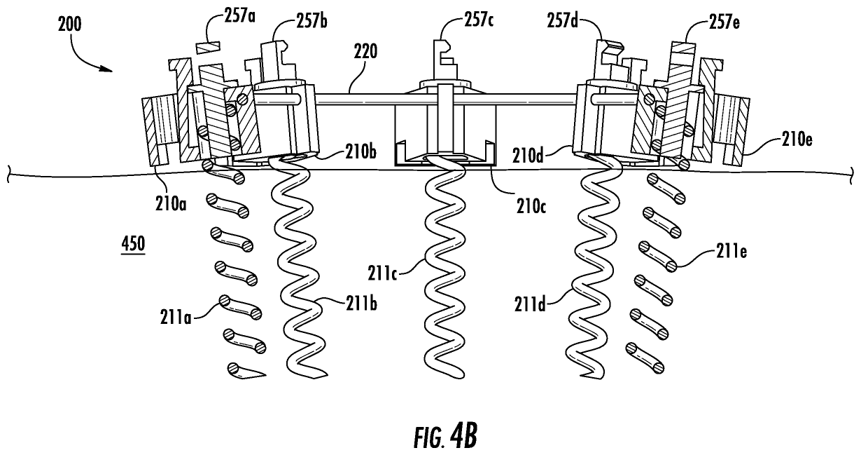

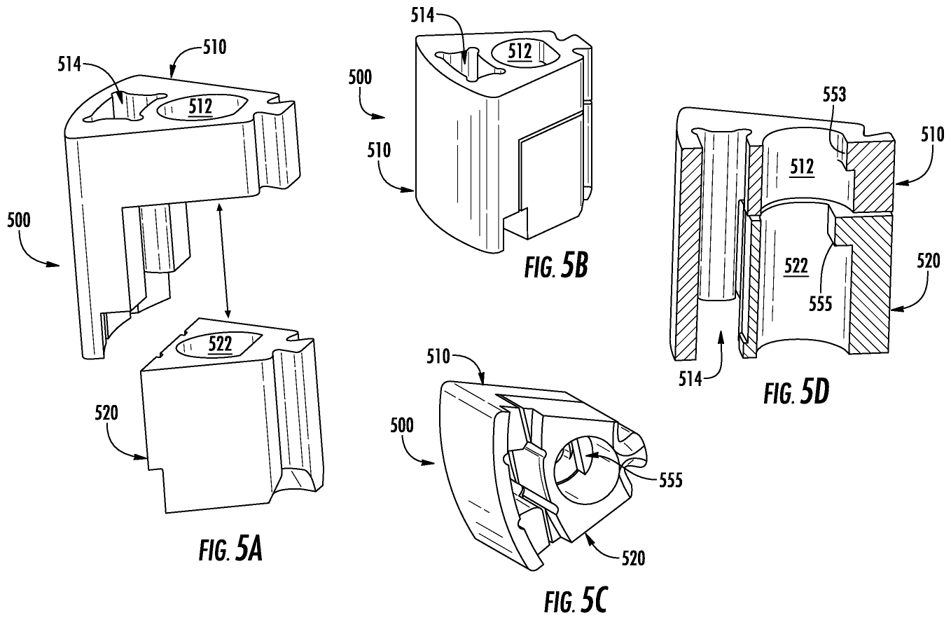

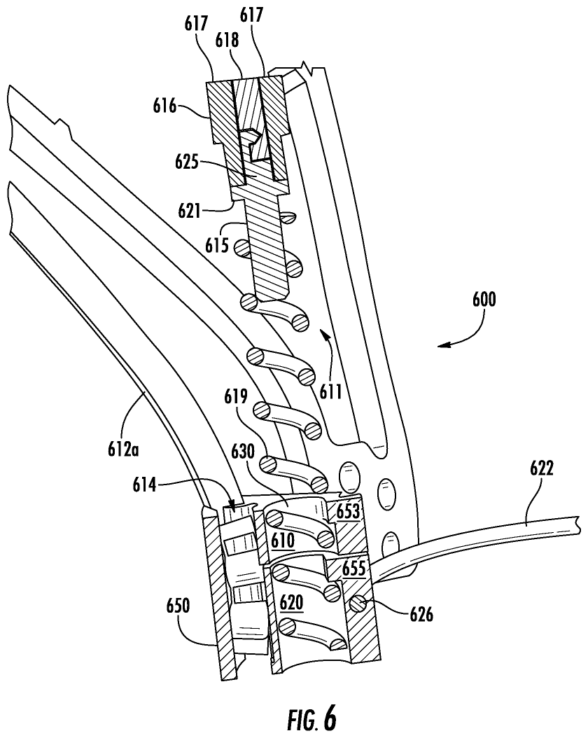

FIGS. 5A-5D provide various perspective views of another embodiment of an anchor housing comprising a retention/release mechanism configured to release annuloplasty components for removal from the cardiac cavity and to retain anchors within the anchor housing following deployment to a treatment site. The anchor housing 500 provides a modular solution that includes a primary anchor housing 510 and a secondary anchor housing 520 which include swages, rails or other features providing fitted engagement of the primary anchor housing with the secondary anchor housing. The primary and secondary anchor housings may be formed from metallic materials and/or polymers with sufficient structural integrity for supporting anchors for driving into the heart annulus. The material may also be chosen based on biocompatibility and fatigue resistance. Material(s) could include stainless steel, Nickel-Titanium, Cobalt-Chromium, Pyrolytic Carbon, Nitinol, polymer materials (e.g., PEEK), and/or other suitable frame materials. In some cases, the anchor sleeves may also be coated with drug-eluting material to prevent fibrosis and/or clotting.

The primary anchor housing 510 may be configured for fixed attachment with a frame or other adjustment mechanism of an annuloplasty system. For example, primary anchor housing 510 is shown to include a frame sleeve 514 sized to fixedly accommodate one or more struts of a frame such as that of FIG. 1. The sleeve 514 may extend through or partially through primary housing 510. The primary housing may include a bore 512 extending therethrough. As shown in FIG. 5D, a ledge 553 may extend at least partially into the bore 512.

The anchor housing 500 may also include a secondary housing 520 comprising a bore 522. The secondary housing may be matingly stacked with the primary housing as shown in FIGS. 5B, 5C and 5D to align the bore 512 with the bore 522. The secondary housing may include a ledge 555 that extends centripetally within bore 522. In one embodiment, a space between the ledge 553 of primary housing 510 and ledge 555 of secondary housing is selected to support one or more turns of a helical anchor, such that the ledges 553 and 555 provide anchor engagement features that guide the anchor during distal translation of the anchor through the bores 512, 522. According to one aspect, the turns of a helical anchor, disposed within the bores 512, 522, couple the primary housing to the secondary housing.

According to one aspect, a centripetal extent of the ledge 553 of the primary housing is smaller than the centripetal extent of the ledge of the secondary housing. A retention feature of the anchor comprises a centripetal extent that enables the retention feature to advance through the bore 512 without interference by ledge 553 but prohibits further distal translation of the retention mechanism into the second bore 522.

FIG. 6 illustrates one embodiment of a portion of an implant system 600 including a modular anchor housing 650 (shown in cross section in FIG. 6) such as those disclosed in FIGS. 5A-5D. The anchor housing 650 includes a primary housing 610 coupled to a secondary housing 620 via an anchor 611. The primary housing 610 may be integral with or fixedly attached to struts 612a of the frame. For example, tabs or flanges 630 included in the anchor housing sleeve 614 of the primary housing may fixedly engage with slots or openings provided in the strut 612a. Alternatively, the primary housing 610 may be molded or otherwise integral with the strut 612a. The proximal end of anchor 611 includes a drive shaft 615 including a drive coupler 625. A helical anchor shaft 619 is coupled to and supported by the drive shaft 615. An anchor retention feature comprising a collar 621 is disposed about the drive shaft between the drive coupler 625 and the helical anchor shaft 619. A driver 616, including a sheath 617 surrounding or partially surrounding a driver 618 may be coupled to the drive coupler 625 to drive the anchor 611 into tissue.

According to one aspect, the collar 621 extends radially outward from the shaft 615. A radial extent of the collar may be small enough to enable the collar to fit within a bore of the primary housing 610 without interference from ledge 653 but large enough to be precluded from entry into the bore of secondary housing 620 by ledge 655. A cinch cord 622 may extend through a cinch lumen 626 disposed in the secondary housing 620 to cinch or otherwise secure anchor housings of the implant.

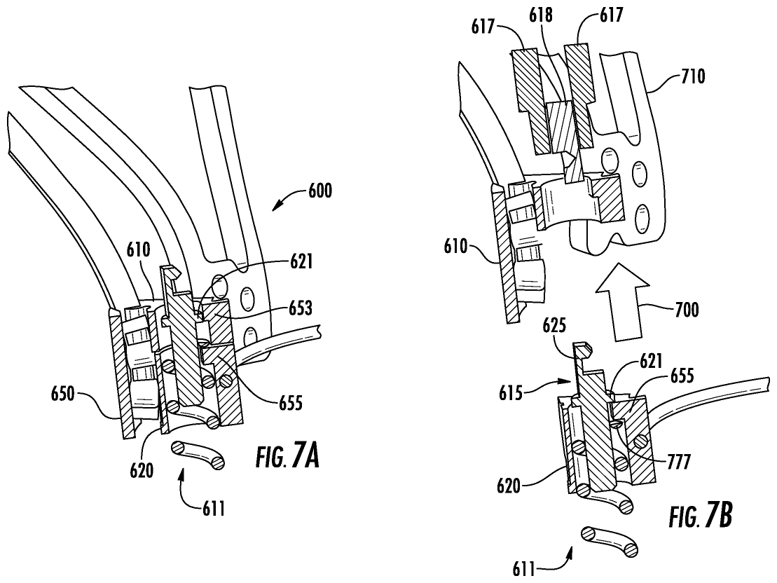

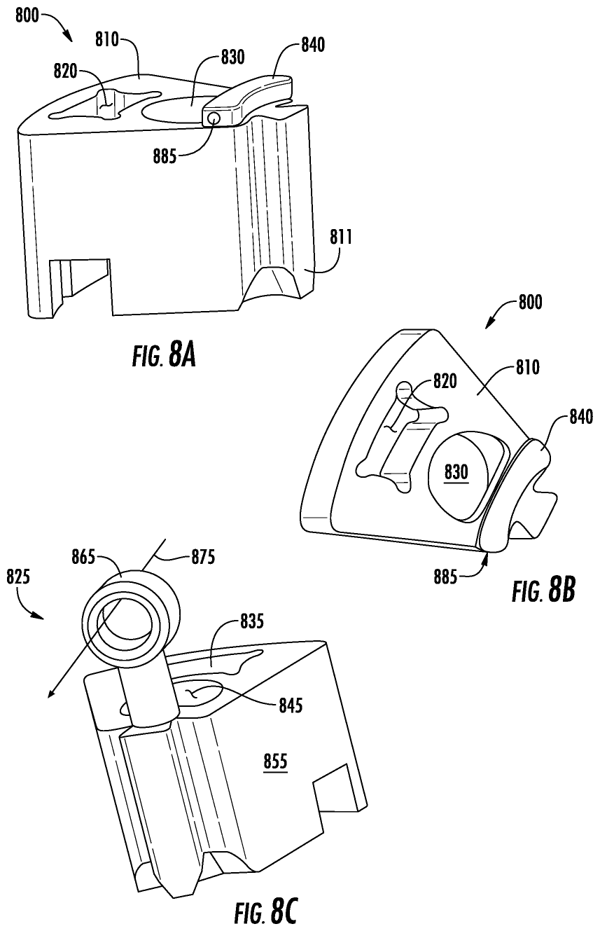

FIG. 7A illustrates the portion of an implant system 600 following translation of the anchor 611 into the primary anchor housing 610. As shown in FIG. 7A, the diameter of collar 621 is smaller than the bore of primary housing 610, and the collar 621 is therefore able to distally translate past ledge 653 until further distal translation of the collar 621 is impeded by the ledge 655 of the secondary bore 620.

In FIG. 7B, the anchor has been distally advanced until further distal travel of the collar 621 is impeded by ledge 655. As shown in FIG. 7B, the initial turn 777 of the anchor 611 engages a distally facing surface of the ledge 655, preventing proximal release of the anchor 611 from the secondary housing 620. Once the anchor 611 is secured by the secondary housing 620, the primary housing 610 (along with coupled frame 710) may be released from the secondary housing 620 by withdrawing the sheath 617 from the distal end of the driver 618 to decouple the driver 618 from the drive coupler 625. Once decoupled, the frame 710 may be withdrawn as indicated by arrow 700 to remove the frame 710, with coupled primary anchor housings 610, from the cardiac cavity.

Accordingly, various embodiments of anchor housings including frame release mechanisms have been shown and described. The anchor housings may be incorporated into annuloplasty systems that include rings and/or expandable frames that may be expanded to a tissue engaging configuration, to secure the anchor housings to the annulus. In some embodiments, after anchoring the implant to the annulus and before removal of the frame, the frame may be used to cinch the annulus to reduce annulus diameter to restore valve competency. Cinching may be achieved though actuation of the frame, for example, by driving collars along struts as discussed with regard to FIG. 1. Other methods for reducing a frame size include sliding collars over struts or threading a cinch cord through the anchor housings or otherwise around the frame and withdrawing the cinch cord until reshaping is achieved. For frames that use actuators or other cinching methods, cinch cords may be used to retain or otherwise bind the anchors in the reshaped configuration.

According to one embodiment, the anchor housings disclosed herein may be modified to include a cinch lumen disposed through at least a portion of an anchor housing. The cinch lumen may support a cinch cord such as cinch cord 220 of FIG. 4B or cinch cord 622 of FIG. 6, either of which may be used to reshape and/or otherwise bind anchor housings of an implant in a reshaped configuration.

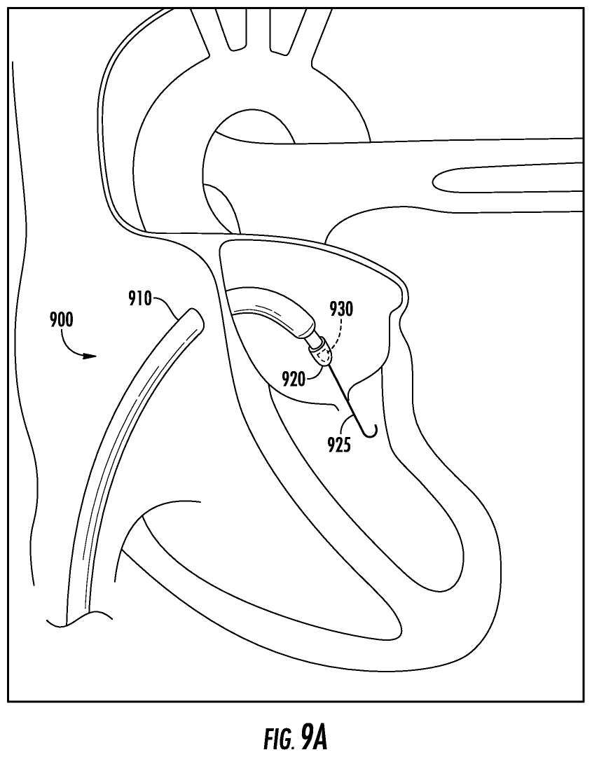

FIGS. 8A and 8B illustrate an anchor housing 800, having an anchor bore 830 configured to support an anchor (not shown) and an anchor housing sleeve 820 configured to support one or more struts of a frame (not shown). A cinch lumen 885 extends through a cinch feature 840, formed on or integral with a body 810 of the anchor housing 800. Although the cinch feature 840 is shown disposed upon a proximal surface of the anchor housing 800, the disclosure is not so limited, and embodiments where the cinch lumen extends through other portions of the anchor housing, such as the flange 811, are considered within the scope of this disclosure.

FIG. 8C illustrates an anchor housing 825, also comprising an anchor bore 845 and an anchor housing sleeve 835 extending through anchor housing body 855. The anchor housing 825 includes an eyelet 865, extending proximally from the body 855 of the anchor housing 825. A cinch lumen 875 is defined by the eyelet 865 of the anchor housing 825. In one embodiment, the eyelet may comprise a swiveling eyelet configured to relieve the stresses and strains from chronic palpitation upon the anchor housing.

Various other methods may be used to cinch the anchors together. For example, in some embodiments the frame may be comprised of a shape memory material which is biased towards frame compression but forced open during anchor implant for proper anchor placement. The present disclosure is not limited to any particular technique for compressing or expanding an implant.

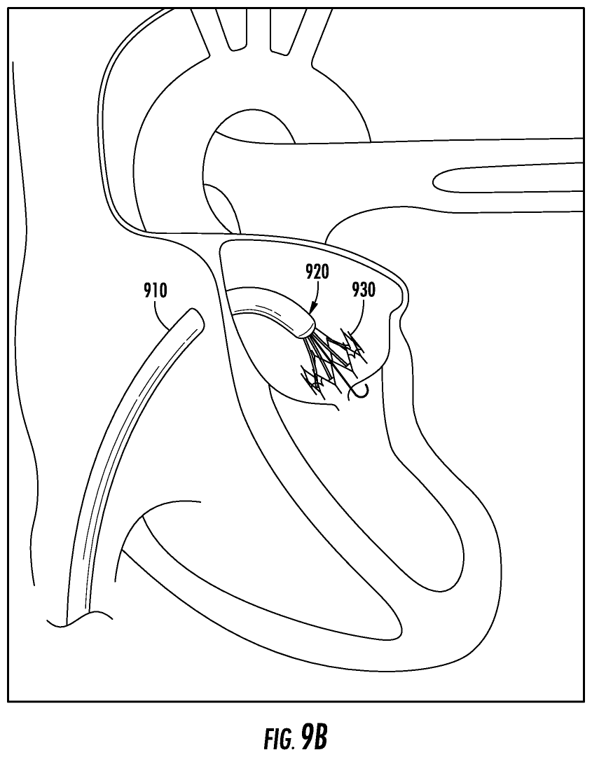

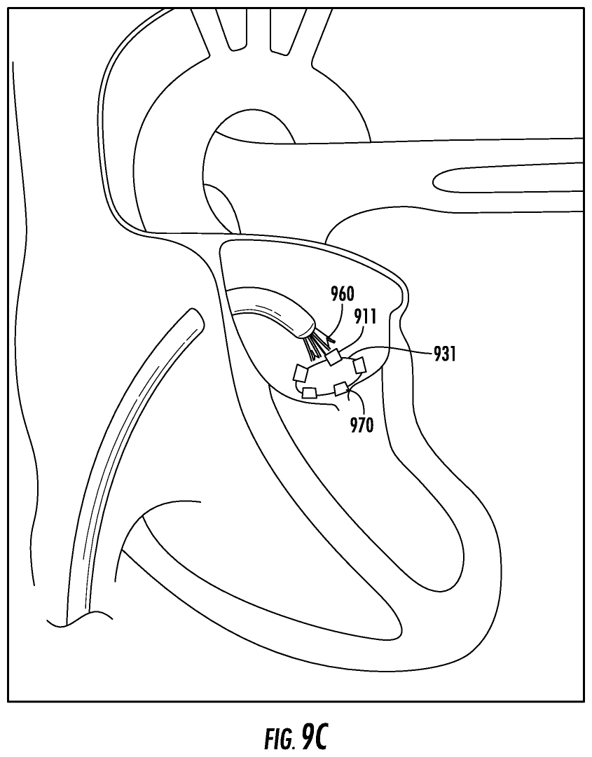

Referring now to FIGS. 9A-9C, an annuloplasty system 900 includes a deployment catheter 910 carrying annuloplasty components 930 at a distal end 920. The distal end of the deployment catheter 910 may be transluminally maneuvered into the left atrium, for example, to position the components above and/or around and/or partially around the mitral valve annulus using guidewire 925. According to one aspect, the deployment catheter may have various positioning and imaging capabilities, for example such as those described in U.S. patent Ser. No. 15/280,004 entitled “Methods for Deployment of Heart Valve Devices Using Intravascular Ultrasound Imaging”, filed Sep. 29, 2016 and incorporated herein by reference.

Referring now to FIG. 9B, when the distal tip 920 of the deployment catheter 910 is appropriately positioned, the annuloplasty components 930 may be exposed (either by advancing the components 930 through the distal tip of deployment catheter 910 or by withdrawing the distal sheath over the components) and expanded to a tissue engaging diameter. Expansion may occur naturally, for example when the frame is formed of Nitinol or other shape memory or super elastic material that is biased towards an expanded state. In alternate embodiments, expansion may be mechanically controlled, for example through the use of a force applied within the frame using an inflatable balloon or the like. The systems and methods disclosed herein are not limited to any particular mechanism for positioning anchors for annular reconstruction, whether such positioning uses a ring or an expandable frame, and for example may use techniques described in U.S. Pat. No. 9,610,156 “Mitral Valve Inversion Prostheses” filed Dec. 24, 2014, U.S. Pat. No. 9,180,005 “Adjustable Endoluminal Mitral Valve Ring”, filed Nov. 24, 2015 and U.S. patent application Ser. No. 15/352,288, entitled “Implantable Device And Deployment System For Reshaping a Heart Valve Annulus” filed Nov. 16, 2016, each incorporated by reference herein.

Referring now to FIG. 9C, following deployment of the annuloplasty components to the heart valve, the annuloplasty components may be cinched or otherwise compressed to adjust the valve annulus to restore valve competency. As described herein, the anchor housings may then release a subset 960 of the annuloplasty components, leaving implant 970 comprising anchors, the anchor housings 911 and the cinch cord 931.

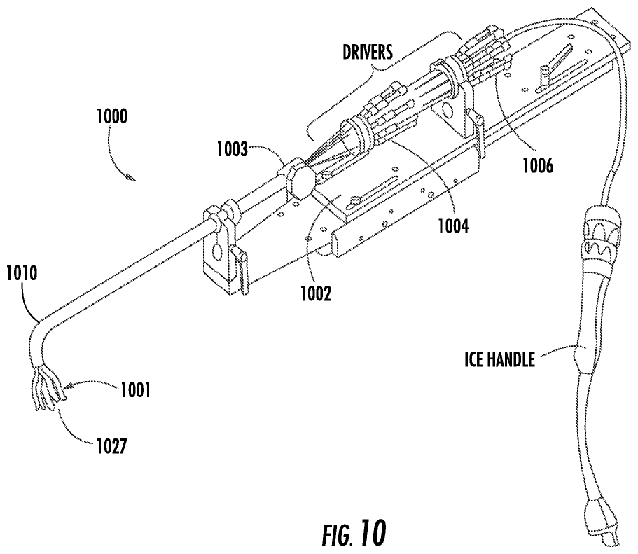

FIG. 10 is a perspective view of an exemplary deployment system 1000 that may be used to deploy an implant 1001 including anchor housings comprising frame release and anchor retention mechanisms as disclosed herein into tissue. The deployment system 1000 comprises a steerable sheath 1010, a sheath steering knob 1003, anchor knobs 1004, cinch knobs 1006, implant 1001, an Intra-Cardiac Echocardiography (ICE) probe 1027, all supported and secured to a base 1002. The cinch knobs 1006 and anchor knobs 1004 may be spring loaded to maintain tension. Rotation of the anchor knobs 1004 may rotationally advance the anchors into the anchor housings and further into annular tissue. Following anchoring, the anchor knobs may be controlled as described to release the frame and/or at least a portion of the anchor housing from the implant 1001.

Accordingly, various embodiments of annuloplasty systems including anchor housings configured to retain and/or release an annuloplasty frame or other components following use have been shown and described. Although embodiments of the present disclosure may be described with specific reference to medical devices and systems (e.g., transluminal devices inserted through a femoral vein or the like) for selective access to heart tissue, it should be appreciated that such medical devices and systems may be used in a variety of medical procedures that require anchoring to heart tissue. The disclosed medical devices and systems may also be inserted via different access points and approaches, e.g., percutaneously, endoscopically, laparoscopically, or combinations thereof.

As used herein, the singular forms “a,” “an,” and “the” are intended to include the plural forms as well, unless the context clearly indicates otherwise. The terms “comprises” and/or “comprising,” or “includes” and/or “including” when used herein, specify the presence of stated features, regions, steps, elements and/or components, but do not preclude the presence or addition of one or more other features, regions, integers, steps, operations, elements, components and/or groups thereof.

As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise.

All numeric values are herein assumed to be modified by the term “about,” whether or not explicitly indicated. The term “about,” in the context of numeric values, generally refers to a range of numbers that one of skill in the art would consider equivalent to the recited value (i.e., having the same function or result). In many instances, the term “about” may include numbers that are rounded to the nearest significant figure. Other uses of the term “about” (i.e., in a context other than numeric values) may be assumed to have their ordinary and customary definition(s), as understood from and consistent with the context of the specification, unless otherwise specified. The recitation of numerical ranges by endpoints includes all numbers within that range, including the endpoints (e.g. 1 to 5 includes 1, 1.5, 2, 2.75, 3, 3.80, 4, and 5).

It is noted that references in the specification to “an embodiment,” “some embodiments,” “other embodiments,” etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described herein, even if not explicitly shown in a particular combination, are nevertheless contemplated as being combinable or arrangeable with each other to form other additional embodiments or to complement and/or enrich the described embodiment(s), as would be understood by one of ordinary skill in the art.

The devices and/or methods disclosed and claimed herein can be made and executed without undue experimentation in light of the present disclosure. While various embodiments of the devices and methods of this disclosure have been described, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

1-22. (canceled)

23. A system for annuloplasty comprising:
a frame having a proximal end, a distal end and struts joined at one of the proximal end or the distal end of the frame; and
an implant comprising:
a plurality of anchors; and
a plurality of anchor housings, at least one anchor housing configured to support and retain one of the plurality of anchors, at least one anchor housing comprising a release mechanism configured to releasably couple the at least one anchor housing to the frame.

24. The system of claim 23 wherein the at least one anchor housing includes:
a bore extending from a proximal end of the at least one anchor housing through the distal end of the at least one anchor housing;
a slot extending longitudinally through a wall of the bore; and
wherein the release mechanism includes a retention tab disposed within the slot, the retention tab comprising a distal foot, a proximal head, and an arm extending from the distal foot to the proximal head, wherein the retention tab includes a first configuration wherein an internal face of the proximal head extends into the bore and a second configuration where the internal face of the proximal head is aligned with the wall of the bore.

25. The system of claim 24 wherein the bore is sized to accept an anchor and the anchor comprises a drive shaft including a proximal connector, a distal anchor shaft coupled to the proximal connector and a collar extending at least partially around the distal anchor shaft.

26. The system of claim 25 wherein the bore and the collar are sized to enable distal translation of the collar within the bore of the anchor housing.

27. The system of claim 26 wherein a first diameter of the proximal connector is less than a second diameter of the collar and distal translation of the collar beyond the proximal head of the retention tab causes the proximal head towards the second configuration to retain the anchor in the anchor housing.

28. The system of claim 26 wherein the proximal head of the retention tab includes a frame engagement feature disposed on an exterior face of the proximal head, the frame engagement feature sized to mate with an anchor housing engagement feature of the frame, and wherein in the first configuration of the retention tab the frame engagement feature engages the anchor housing engagement feature, and in the second configuration the frame engagement feature releases the anchor housing engagement feature to release the frame from the implant.

29. The system of claim 23 wherein the release mechanism of the at least one anchor housing includes:
a primary housing coupled to the frame; and
a secondary housing releasably coupled to the primary housing by an anchor.

30. The system of claim 29 wherein the primary housing comprises a primary bore extending therethrough and a first anchor engagement feature disposed on a primary bore wall, and the secondary housing includes a secondary bore extending therethrough and a second anchor engagement feature disposed on a secondary bore wall, the second anchor engagement feature extending further centripetally within the secondary bore than the first anchor engagement feature extends within the primary bore.

31. The system of claim 30 wherein the primary housing comprises a proximal end and a distal end and the secondary housing is configured to matingly engage with the distal end of the primary housing to align the primary bore with the secondary bore.

32. The system of claim 31 wherein the primary bore and secondary bore are sized to support the anchor, and the anchor comprises a drive shaft including a proximal connector, a distal anchor shaft and a collar extending at least partially around the distal anchor shaft, the distal anchor shaft including at least two external engagement features, each configured to engage at least one of the primary anchor engagement feature or the secondary anchor engagement feature.

33. The system of claim 32 wherein a first centripetal extent of the first anchor engagement feature is configured to enable distal advancement of the collar of the anchor into the primary bore, and a second centripetal extent of the second anchor engagement feature is configured to preclude distal advancement of the collar into the secondary bore.

34. The system of claim 23 wherein a subset of anchor housings each include a lumen extending therethrough, and wherein the implant further comprises:
a cinch cord extending through the lumen of the subset of anchor housings to join the subset of anchor housings.

35. The system of claim 34 wherein the subset of anchor housings comprises a primary housing and a secondary housing, and the lumen extends through the secondary housing.

36. An implant comprising:
a plurality of anchors;
at least two anchor housings, each anchor housing comprising:
a release mechanism configured to releasably couple the anchor housing to an implant delivery system; and
a cinch lumen extending through a portion of the anchor housing; and
a cinch cord extending through the cinch lumen of the at least two anchor housings to join the at least two anchor housings.

37. The implant of claim 36 wherein at least one anchor housing includes:
a bore extending from a proximal end of the at least one anchor housing through a distal end of the at least one anchor housing;
a slot extending longitudinally through a wall of the bore; and
wherein the release mechanism includes a retention tab disposed within the slot, the retention tab comprising a distal foot, a proximal head, and an arm extending from the distal foot to the proximal head, wherein the retention tab includes a first configuration wherein an internal face of the proximal head extends into the bore and a second configuration where the internal face of the proximal head is aligned with the wall of the bore.

38. The implant of claim 37, wherein the implant delivery system comprises a frame and wherein the proximal head of the retention tab includes a frame engagement feature disposed on an exterior face of the proximal head, the frame engagement feature sized to mate with an anchor housing engagement feature of the frame, and wherein in the first configuration of the retention tab the frame engagement feature engages the anchor housing engagement feature, and in the second configuration the frame engagement feature releases the anchor housing engagement feature to release the frame from the implant.

39. The implant of claim 38 wherein the implant delivery system includes a frame and the release mechanism of the at least one anchor housing includes a primary housing coupled to the frame and a secondary housing releasably coupled to the primary housing by an anchor, and the cinch lumen is provided by the secondary housing.

40. An annuloplasty method including the steps of:
deploying an implant system to a valve annulus, the implant system comprising a frame and an implant comprising a plurality of anchors translatably supported by a plurality of anchor housings, the plurality of anchor housings releasably coupled to the frame by a release mechanism of each anchor housing;
securing the implant to the valve annulus by driving the plurality of anchors at least partially through the plurality of anchor housings and into the valve annulus;
cinching the valve annulus using a cinching mechanism of the frame;
releasing the frame from the plurality of anchor housings using the release mechanism of each anchor housing; and
withdrawing the frame from the valve annulus.

41. The method of claim 40, wherein at least one anchor housing includes a bore extending from a proximal end of the at least one anchor housing through a distal end of the at least one anchor housing, and the release mechanism of each anchor housing includes a retention tab disposed within the bore and having a frame engagement feature configured for mated engagement with an anchor engagement feature of the frame when the retention tab is in an unbiased state, and wherein the step of releasing the frame from the plurality of anchor housings includes the steps of distally translating an anchor through the at least one anchor housing until the retention tab returns to a biased state to release the frame engagement feature from the anchor engagement feature.

42. The method of claim 40, wherein at least one anchor housing includes a primary housing coupled to the frame and a secondary housing releasably coupled to the primary housing by an anchor, the release mechanism of each anchor housing comprises a first anchor engagement feature disposed within a primary bore of the primary housing and a second anchor engagement feature disposed within a secondary bore of the secondary housing, the second anchor engagement feature extending further centripetally within the second bore than the first anchor engagement feature extends within the primary bore, and wherein the step of releasing the frame from the plurality of anchor housings includes the step of distally translating the plurality of anchors through the primary housings and the secondary housings until proximal anchor collars of the plurality of anchors extend into the secondary housing and are precluded from further distal translation by the second anchor engagement feature.
